(19) United States
(12) Patent Application Publication
(10) Pub. No.: US 2020/0176106 A1
(43) Pub. Date: Jun. 4, 2020

(54) Hospital clinical workforce redeployment system and method of use

(71) Applicant: Alexander Pieroni, Newton, NJ (US)

(72) Inventor: Alexander Pieroni, Newton, NJ (US)

(21) Appl. No.: 16/602,710

(22) Filed: Nov. 25, 2019

Related U.S. Application Data
(60) Provisional application No. 62/917,263, filed on Nov. 30, 2018.

(51) Int. Cl. G16H 40/20; G06Q 10/06; G06Q 10/10; G06Q 40/08

(52) U.S. Cl. CPC G16H 40/20; G06Q 10/063118; G06Q 10/0633; G06Q 40/08; G06Q 10/06398; G06Q 10/105

(57) ABSTRACT

A status and workflow management system, analytic tools, artificial intelligence, and monitoring indexes along with a status and workflow management system, including an intake mechanism and computer systems for use by responsible persons responsible for respective parts of the processing of individual telehealth workforce redeployment opportunities for the at-risk clinical employee populations at a hospital, interests in monitoring hospital telehealth initiatives, and or evaluating the utilization of at-risk clinical employees within telehealth initiatives. A system comprising at least two computers, a telemetric data collection system, and artificial intelligence providing a mechanism that generates for each responsible person a workflow status screen and monitoring dashboards comprising of information pertaining to the given responsible person's perspective of involvement. A task generator element capable of displaying to the given responsible person on his or her screen tasks to which he or she has been assigned and or self-elected to do so. A task update mechanism updates tasks on the screen of the given responsible person in accordance with a sequence of workflow steps. An automatic mechanism for displaying monitoring indexes relative to fluctuations in data. An automatic mechanism for updating reporting and decision making tools via evolving artificial intelligence applied predictive modeling.

### BACKGROUND OF THE INVENTION

### Field of the Invention

Business productivity tool providing a workforce redeployment system utilizing “at-risk” (disabled, injured, aging, etc) clinical employee populations within hospital systems.

### Background Art

Clinical employees working within hospitals systems need to be able to perform ‘at or near full physical function capacity’ in order to perform within their very physically demanding job description expectations.

Over time this creates an ever growing pool of “At Risk” clinical employees that are not able to contribute to a hospital's core job performance expectations. There are many reasons a clinical employee might become ‘at-risk’, this includes those who develop disabilities, injuries, illnesses, require surgery, maternity leave related needs, limitations related to the natural aging cycle, career burn-out, etc. According to the Depart of Labor, Hospital based nursing is the number one non-fatal work injury classification category.

Hospitals pay insurance carriers to insure this at-risk employee population, substituting employee income with claim payments until these employees are able to return to ‘full-duty’ at their job. Current processes are designed to evaluate an employee's physical functional capacity relative to being able to carry out their core employment responsibilities of the job they held at the time their disability presented.

Typical costs include loss of work for the employee (Harvard estimates 63% of all bankruptcies are due to a medical issue); the high cost of temporary staff to replace the employee/s out on an LOA (leave of absence); the high cost of insurance policies—ie—Workers Compensation Insurance, Short Term Disability Insurance, and Long Term Disability Insurance.

The algorithms and calculation formulas currently utilized by the insurance carriers to evaluate risk, predict cost, and dictate claim management strategies for the affected hospitals and their employees have not changed significantly for decades.

### Advantages of Current Invention

With the emergence of new telehealth legislation, technologies, and telehealth insurance reimbursement trends, successful transitions into telehealth roles can offer significant opportunities for health systems to impact their costs relative to staffing various telehealth initiatives, as well as offer significant cost reductions to insurance premiums paid—ie—workers compensation & disability plans.

Current Hospital HR policies and procedures were designed prior to the many advances in legislation, technology and telehealth reimbursement codes, where it now makes it unnecessary, in many cases, to make granular evaluations of an employee's physical function capacity.

Although patient demand for telehealth exists in the marketplace, hospital's are not cultivating a workforce to meet this demand, and instead 3rd party telemedicine networks have organized to compete with hospitals, disrupting the continuity of patient care teams.

Currently, the connection of information and data between telehealth, hospital, and insurance does not exist. Utilizing the invention to pull data from a multitude of sources; the invention tracks, interprets, learns, predicts, and makes decision support data available to stakeholders—ie—disability and workers compensation insurance carriers, hospital human resource departments, hospital management teams, etc.

This data also becomes useful to the many vendors and departments being affected by a hospital system's telehealth initiative decision making process where budgets are constrained and many hospitals rely on 3rd party telemedicine networks to fulfill their needs which drives up cost and disconnects patients from their local health system resources.

By empowering Hospital Human Resources with tools that translate thousands of data points into meaningful employee-specific quantifying telehealth strategies, everyone benefits—from the employees who are able to transition into a meaningful employment capacity and avoid financial strains due to loss of income; cost savings and new telehealth revenue for the hospital system; Insurance Carriers via reduced claims.

Hospital human resource departments, hospital-centric telemedicine initiatives, and Insurance Carrier's Insuring the hospital At-Risk populations, now all become connected via a collaborative utilization strategy that provides all parties with aligned financial incentives and risk-reducing methods.

The invention would make it possible for human resources to be connected with their disability and workers compensation insurance carriers by providing ‘real-time’ decision support. The invention is also using artificial intelligence to make logical decision opportunities between supply and demand. SUPPLY: the work potential of clinical employees at-risk of going on a disability claim relative to their education, work experience, and personal physical diagnosis (no longer able to perform at their core job function, but qualified to participate in the system invention) and DEMAND: the current underserved patients of the hospital that could benefit from telemedicine programs as per the definitions of each patient's health insurance reimbursement source—ie—payors like Medicare, Medicaid, BCBS, etc.

### DESCRIPTION OF THE DRAWINGS

FIG. 1 Demonstrates the data flow between Telehealth Programs, Human Resources, Carriers & Brokers, and the invention's artificial intelligence processing source. The bottom of the drawing represents the clinical employee resource pool where employees will present as sick, injured, aged, etc. and become eligible for redeployment.

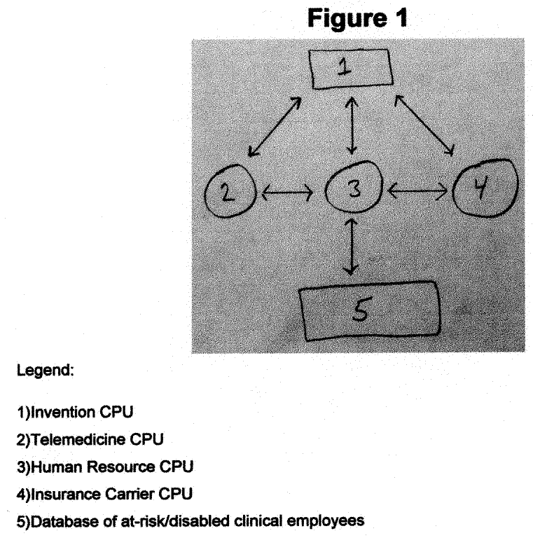

FIG. 2 Demonstrates the integration of invention tools and indexes into existing popular dashboards likely already in-use at hospital HR and Carrier clients for purposes that could be related to monitoring employees—ie—payroll, tracking PTO, tracking absences, sick leave etc. “1” represents the invented utility.

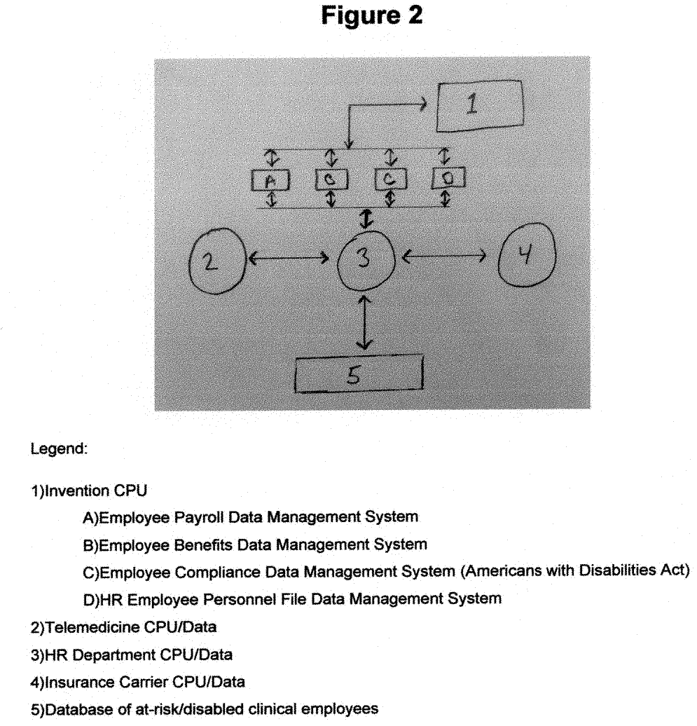

FIG. 3 is a stylized depiction of the hardware to implement a workforce redeployment system utilizing the ‘at-risk’ employee populations within a hospital system, with an explanatory legend.

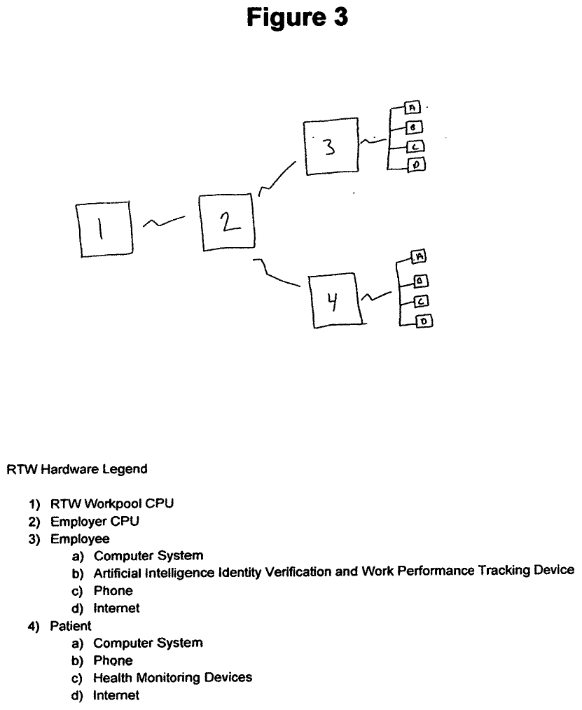

FIG. 4 is a high-level overview of the novel processes that compare the ‘present invention’ relative to the processes that exist within the industry's ‘prior art’ standard of current operation.

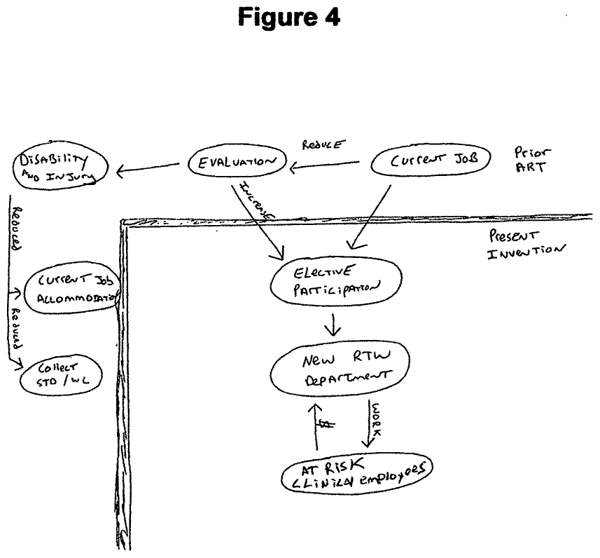

FIG. 5 represents a computer dashboard screen as would be utilized by a hospital HR department depicting a hospital's total at-risk clinical employee population along with redeployment utilization decision support.

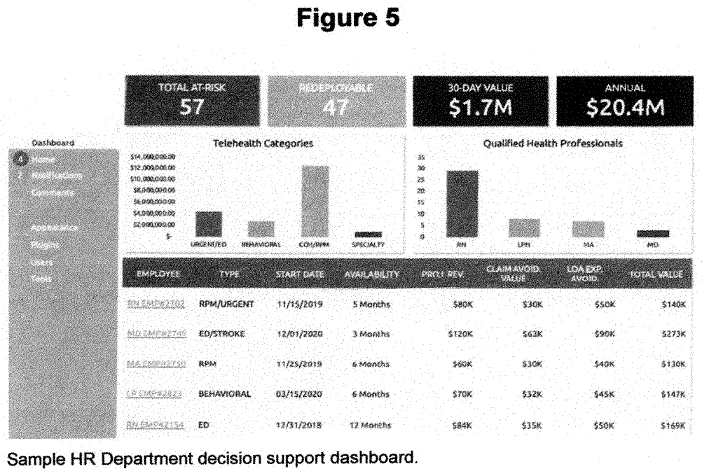

### SUMMARY OF THE INVENTION

Predictive tools and artificial intelligence to assist those who are either 1) involved with the processes of developing and or staffing hospital-centric telehealth initiatives. 2) involved with the compliance and or management of hospital employees that present with at-risk scenarios—ie—disabilities, injuries, needs for surgeries, maternity, limitations due to aging, etc. 3) involved with evaluating the overall risk and or compliance equations related to a hospital's ‘at-risk’ population. 4) involved with the overall risk evaluation of a hospital or hospital system relative to insuring a hospital system's ‘at-risk’ employee populations for the purposes of insurance products that include Workers Compensation Insurance, Short Term Disability Insurance, and Long Term Disability Insurance.

### DETAILED DESCRIPTION OF THE INVENTION

The present invention is a system composed of computer hardware, computer software, telemetry device, and a communication device.

In a preferred embodiment, the present invention is deployed at a hospital system employer location, and or on hospital employer assets, and or with a company responsible for managing the risk of a hospital's clinical employee population—ie—insurance carriers, third party administrators, insurance brokers, hospital trusts, etc.

ANALYTIC TOOLS: Algorithms for the insurance company's evaluation of risk and the cost of risk; Decision Support for hospital installed RTW (return to work) programs for the ‘at-risk’ clinical employee population; Financial planning tools assisting those responsible for managing hospital based clinical employee populations.

EMPLOYEE AT-RISK POPULATION UTILIZATION INDEX: Provides performance measurement insight to those concerned with managing employee risk. (HR, Compliance, Legal, Brokers, Carriers, Hospital Trusts, Risk Advisors etc). Similar to what “FICO” scores mean to a financial lender, these indexes evaluate each hospital based on their effectiveness in utilizing their at-risk employees thereby avoiding or reducing insurance claims. This INDEX can have the result of an insurance company being willing to offer more competitive pricing on their workers compensation and disability products, and or price higher-risk hospital systems accordingly so as to retain acceptable ROI (return on investment) ratios relative to their quoted premium costs.

DASHBOARDS: Provides Decision support and reporting value to stakeholders—ie—insurance carriers, human resource departments at hospitals, telemedicine department managers etc.

HOSPITAL GRADING INDEX AND PROFILE REPORTING FOR RFP OR BIDDING NEW BUSINESS PURPOSES: Provides Brokers, Hospital Trusts, and Insurance Carriers with highly relevant comparative data, resulting in more accurately classified hospitals for the purposes of their sales teams/efforts involved with bidding on new business and or retaining existing clients.

By empowering Human Resources with connective tools that translate thousands of data points into meaningful employee-specific quantifying telehealth strategies, everyone benefits—from the employee who temporarily transitions to, and or, begins a new career without having to leave their hospital; the Hospital Financial Budget via many cost savings and new telehealth revenue; Brokers and Carriers via higher client retention and new client expansion; improved and expanded patient care via telehealth; less at-risk clinical employee claimants experiencing losses of personal income.

Relative to future compliance with trending labor laws, and supporting the embodiment of the ADA (Americans with Disabilities Act), the invention makes it possible for hospitals and large health systems to measure a clinical employee's work ‘ability’ potential v.s. their ‘disability’ limitations. The invention tools support the very spirit of the Americans with Disabilities Act by encouraging more “reasonable accommodations’ be made available to at-risk clinical hospital employees (disabilities, injuries, natural limitations associated with aging, etc) via the benefit of the invention's ability to empower the collaboration of the many entities responsible for managing a hospital system's human capital resources.

I claim:

1) A status, measurement index(s), artificial intelligence empowered management tools, and data exchange for the at-risk clinical employees of hospital health systems comprising
a) At least two computers,
b) A profile of at-risk employees,
c) A profile of hospital systems,
d) A profile of telehealth initiatives,
e) A database of open projects,
f) A database of insurance classification(s),
g) An assignment element capable of matching employees to at least one open project,
h) A task generator element,
i) An update mechanism for each said task,
j) A tracking element associated with each task of a project,
k) A telemetric data collection means for monitoring said employee performance by said task;
l) A reporting element to communicate said performance to parties involved with managing employees, responsible for managing risk, insurers, data collectors.
The status and workflow-management system of claim 1, further comprising a cost recoupment element for said employee's employer.
The status and workflow-management system of claim 1, further comprising input from at least one external carrier.
